(19) United States
(12) Patent Application Publication
(10) Pub. No.: US 2025/0367095 A1
(43) Pub. Date: Dec. 4, 2025

(54) COMPOSITIONS AND METHODS FOR CLEANSING KERATIN MATERIALS

(71) Applicant: L'Oreal, Paris (FR)

(72) Inventors: Shilpa ARORA, South Planfield, NJ (US); Zavvia Ramos, Jersey City, NJ (US); Aziza Khader Suleiman, Paterson, NJ (US)

(21) Appl. No.: 18/731,070

(22) Filed: May 31, 2024

(51) Int. Cl. A61K 8/46; A61K 8/42; A61K 8/49; A61Q 5/02

(52) U.S. Cl. CPC A61K 8/466; A61K 8/42; A61K 8/4926; A61Q 5/02; A61K 2800/30; A61K 2800/5422; A61K 2800/5424; A61K 2800/5428

(57) ABSTRACT

The disclosure relates to compositions having (a) a surfactant system comprising (i) at least one first anionic surfactant, (ii) optionally at least one second anionic surfactant different from the first anionic surfactant, and (iii) optionally at least one amphoteric surfactant, (b) at least one scalp benefit agent, and (c) water, wherein the compositions are free or essentially free of sulfate-based surfactants, as well as to methods of using the compositions. The compositions are cleansing compositions that can be used in methods of cleansing keratin materials, such as hair.

### TECHNICAL FIELD

The disclosure relates to compositions for cleansing keratin materials, and to methods of using the compositions. The compositions are free or essentially free of sulfate-based surfactants.

### BACKGROUND

Personal care cleansing compositions such as shampoo, body wash, facial cleanser, etc., use anionic cleansing surfactants to remove sebum and exogenous contaminants such as dirt, makeup, styling products, etc., from the surface of keratinous materials such as skin and hair. When present at concentrations higher than the critical micelle concentration, the majority of the surfactant molecules self-assemble into micelles that have a highly negative surface charge, enabling the surfactants to solubilize hydrophobic components into the aqueous phase of the composition, which are then removed from the keratinous material.

Typically, personal care cleansing compositions use sulfate-based anionic surfactants such as sodium lauryl sulfate (SLS) or sodium laureth ether sulfate (SLES). These surfactants are commonly used because they have good foaming and cleansing properties, permit the composition to be thickened easily to achieve a desirable viscosity, and are relatively inexpensive. However, there have been growing concerns in the marketplace over the negative effects of these or other sulfate-based surfactants on the skin and body. For example, sulfate-based surfactants have a tendency to dry out hair and skin, strip dye from color-treated hair, and may cause skin and eye irritation. In addition, SLES may contain dioxanes, byproducts generated in the manufacturing process, which are considered carcinogenic at high enough levels. As such, cleansing compositions for the skin and hair that are free or essentially free of sulfate-based surfactants are becoming increasingly desirable to consumers.

However, there are challenges in developing suitable personal care cleansing formulations without sulfate-based surfactants. For example, most existing sulfate-free personal care cleansing products foam poorly, which is considered a significant drawback by consumers. Further, cleansing compositions containing anionic surfactants that are not sulfate-based are not easily thickened. Traditional methods of increasing viscosity of these formulations are not effective with sulfate-free surfactants. In addition, some anionic cleansing surfactants which are effective cleansers are undesirable for use in personal care compositions, as their harsh nature will strip the hair or skin of natural components that are necessary for healthy function, for example negatively impacting the skin's barrier integrity. Moreover, certain non-sulfate cleansing surfactants are ineffective for depositing care components such as moisturizing agents onto the treated keratin materials.

Thus, there is a need to develop personal care cleansing compositions that are free or substantially free of sulfate-based surfactants, foam adequately, have a desirable viscosity, and provide advantageous benefits to the keratin materials. However, this requires careful balancing of components to ensure the properties of both the cleansing composition and the properties imparted to the keratin materials meet consumers' needs. However, such balance has been difficult to achieve to date, as efforts to achieve certain properties of the cleansing composition such as foaming or thickness have interfered with efforts to achieve certain cosmetic properties imparted by the composition, such as moisture or hydration.

It has now been found that, by using a unique combination of surfactants, shampoo compositions that are free or essentially free of sulfate-based surfactants can be prepared which surprisingly have abundant, thick, and creamy foam and desirable viscosity, while also providing excellent cosmetic properties to hair such as detangling, ease of application, luxurious hair feel, volume, bounce, manageability, and/or frizz control, and which are able to provide additional benefits to the scalp.

### SUMMARY

The disclosure relates to compositions for cleansing keratin materials, and to methods for using the compositions. The compositions are free or essentially free of sulfate-based surfactants, yet surprisingly generate abundant foam.

In various embodiments, the compositions comprise (a) a surfactant system comprising (i) at least one first anionic surfactant chosen from sulfosuccinates, sulfoacetates, acyl sarcosinates, and/or salts thereof, (ii) optionally at least one second anionic surfactant different from the first anionic surfactant, and (iii) optionally at least one amphoteric surfactant, (b) at least one scalp benefit agent, and (c) water. The surfactant system optionally further comprises (iv) at least one nonionic and/or cationic surfactant. The compositions optionally further comprise additional components such as conditioning agents, thickening agents, and/or auxiliary agents. The compositions are free or essentially free of sulfate-based surfactants.

In further embodiments, the compositions comprise (a) a surfactant system comprising (i) at least one first anionic surfactant chosen from sulfosuccinates, sulfoacetates, acyl sarcosinates, and/or salts thereof, (ii) optionally at least one second anionic surfactant different from the first anionic surfactant, (iii) optionally at least one amphoteric surfactant, and (iv) optionally at least one nonionic surfactant, (b) at least one scalp benefit agent, (c) water, and (d) optionally at least one additional component chosen from conditioning agents, thickening agents, and/or auxiliary agents such as anti-dandruff active agents. The compositions are free or essentially free of sulfate-based surfactants. In various embodiments, the compositions comprise at least one second anionic surfactant chosen from alkyl sulfonates, sulfosuccinates, sulfoacetates, salts thereof, or combinations of two or more thereof, for example olefin sulfonates such as C10-C24 olefin sulfonates and/or salts thereof. In embodiments where the compositions include an amphoteric surfactant, the total amount of anionic surfactants may, for example, range from about 0.5% to about 15% by weight, relative to the total weight of the composition. Optionally, the composition comprises at least one amphoteric surfactant chosen from betaines, alkyl sultaines, alkyl amphoacetates, amphopropionates, salts thereof, or combinations of two or more thereof, and is optionally free or essentially free of cocamidopropyl betaine. In embodiments where the compositions include an amphoteric surfactant, the compositions may have a weight ratio of the total amount of anionic surfactant(s) to the total amount of amphoteric surfactant(s) that is less than about 10:1, such as less than about 9:1, less than about 8:1, less than about 7:1, less than about 6:1, or less than about 5:1, for example may range from about 1:1 to about 9:1, about 1:1 to about 8:1, about 1:1 to about 7:1, about 1:1 to about 6:1, about 1:1 to about 5:1, about 1:1 to about 4:1, or about 1:1 to about 3.5:1. In various embodiments, the compositions comprise at least one conditioning agent chosen from cationic conditioning agents, silicone compounds, non-silicone fatty compounds, or combinations thereof. In various embodiments, the compositions comprise at least one polysaccharide-based thickening agent.

In still further embodiments, the compositions comprise (a) a surfactant system comprising (i) at least one first anionic surfactant chosen from sulfosuccinates, sulfoacetates, acyl sarcosinates, and/or salts thereof, (ii) optionally at least one second anionic surfactant chosen olefin sulfonates such as C10-C24 olefin sulfonates, and/or salts thereof, (iii) optionally at least one amphoteric surfactant, and (iv) optionally at least one nonionic surfactant, (b) at least one scalp benefit agent, (c) water, and (d) at least one additional component chosen from conditioning agents, thickening agents, and/or auxiliary agents such as anti-dandruff active agents. In embodiments where the compositions include an amphoteric surfactant, the compositions may have a weight ratio of the total amount of anionic surfactant(s) to the total amount of amphoteric surfactant(s) that is less than about 10:1, such as less than about 9:1, less than about 8:1, less than about 7:1, less than about 6:1, or less than about 5:1, for example may range from about 1:1 to about 9:1, about 1:1 to about 8:1, about 1:1 to about 7:1, about 1:1 to about 6:1, about 1:1 to about 5:1, about 1:1 to about 4:1, or about 1:1 to about 3.5:1. The compositions are free or essentially free of sulfate-based surfactants and optionally free or essentially free of cocamidopropyl betaine.

In further embodiments still the compositions comprise (a) a surfactant system comprising (i) at least one first anionic surfactant chosen from disodium laureth sulfosuccinate, sodium lauryl sulfoacetate, sodium lauroyl sarcosinate, or combinations of two or more thereof, (ii) optionally at least one second anionic surfactant chosen from olefin sulfonates such as C10-C24 olefin sulfonates, and/or salts thereof, for example sodium C14-C16 olefin sulfonate, (iii) optionally at least one amphoteric surfactant chosen from cocamidopropyl hydroxysultaine, disodium cocoamphodiacetate, cocobetaine, or combinations of two or more thereof, and (iv) optionally at least one nonionic surfactant, (b) at least one ceramide, (c) water, and (d) at least one additional component chosen from conditioning agents, thickening agents, and/or auxiliary agents such as anti-dandruff active agents. In embodiments where the compositions include an amphoteric surfactant, the compositions may have a weight ratio of the total amount of anionic surfactant(s) to the total amount of amphoteric surfactant(s) that is less than about 10:1, such as less than about 9:1, less than about 8:1, less than about 7:1, less than about 6:1, or less than about 5:1, for example may range from about 1:1 to about 9:1, about 1:1 to about 8:1, about 1:1 to about 7:1, about 1:1 to about 6:1, about 1:1 to about 5:1, about 1:1 to about 4:1, or about 1:1 to about 3.5:1. The compositions are free or essentially free of sulfate-based surfactants and optionally free or essentially free of cocamidopropyl betaine.

The disclosure further relates to methods of using the compositions, the methods comprising applying the compositions to keratin materials and rinsing the keratin materials. For example, the compositions may be shampoo compositions and the methods may be methods of cleansing hair.

### BRIEF DESCRIPTION OF THE FIGURES

FIG. 1 shows a comparison of various properties of hair subjected to a shampoo and conditioner routine using a sulfate-free shampoo composition according to the disclosure compared to a commercially-available sulfate-free shampoo composition.

FIG. 2 is an image of foam generated by a sulfate-free shampoo composition according to the disclosure (1E) compared to a commercially-available sulfate-containing shampoo composition (C2).

FIG. 3 is an image of foam generated by a sulfate-free shampoo composition according to the disclosure (1E) compared to a commercially-available sulfate-containing shampoo composition (C2).

FIGS. 4A-4B are graphs of height measurements of foam generated by a sulfate-free shampoo composition according to the disclosure (FIG. 4A) and foam generated by a commercially-available sulfate-containing shampoo composition (FIG. 4B).

FIG. 5 shows images of hair swatches cleansed with shampoo compositions according to the disclosure.

### DETAILED DESCRIPTION

The disclosure relates to compositions for cleansing keratin materials, and to methods for cleansing keratin materials with the compositions. The compositions are free or essentially free of sulfate-based surfactants, yet surprisingly have good foaming properties, and provide desirable cosmetic benefits to the treated keratin materials such as the scalp and/or hair.

### I. Compositions

Compositions according to the disclosure include (a) a surfactant system comprising (i) at least one first anionic surfactant, (ii) optionally at least one second anionic surfactant different from the first anionic surfactant, and (iii) optionally at least one amphoteric surfactant, (b) at least one scalp benefit agent, and (c) water. The surfactant system may optionally further comprise one or more nonionic surfactants and/or cationic surfactants, and the compositions may optionally comprise additional components such as conditioning agents, thickening agents, active agents, emulsifiers, and auxiliary components. The compositions are free or essentially free of sulfate-based surfactants.

### Surfactant System

Compositions according to the disclosure comprise a surfactant system that includes combinations of surfactants that are not sulfate-based. The combination of certain types and amounts of surfactants surprisingly provides advantageous properties to the compositions and permits successful deposition onto the keratin materials, such as the scalp and/or hair fibers, of components that provide advantageous benefits thereto.

### Anionic Surfactants

Compositions according to the disclosure comprise at least one first anionic surfactant chosen from sulfosuccinates, sulfoacetates, acyl sarcosinates, and/or salts thereof, and optionally at least one second anionic surfactant different than the first anionic surfactant. Salts of anionic surfactants are expressly included, whether or not stated.

In some embodiments, the second anionic surfactant comprises one or more sulfo-derived surfactants, and in some embodiments the second anionic surfactant comprises surfactants that are not sulfo-derived. For example, the second anionic surfactant may comprise one or a combination of sulfo-derived anionic surfactants, may comprise a combination of sulfo-derived and non-sulfo-derived anionic surfactants, or may comprise one or a combination of anionic surfactants that are not sulfo-derived.

Sulfo-derived surfactants are understood to be surfactant compounds containing a sulfonate group. Exemplary useful sulfo-derived surfactants include but are not limited to sulfosuccinates, sulfoacetates, isethionates, and alkyl sulfonates, which include, for example, alkyl aryl sulfonates, aryl alkyl sulfonates, alkyl ester sulfonates, and olefin sulfonates. For example, primary alkane disulfonates, alkene sulfonates, hydroxyalkane sulfonates, alkyl glyceryl ether sulfonates, sulfonates of alkylphenolpolyglycol ethers, alkylbenzenesulfonates, phenylalkanesulfonates, alkene sulfonates, hydroxyalkanesulfonates and disulfonates, secondary alkanesulfonates, paraffin sulfonates, ester sulfonates, sulfonated fatty acid glycerol esters, and/or alpha-sulfo fatty acid methyl esters including methyl ester sulfonate may be chosen.

Useful and non-limiting alkyl sulfonates include those of formula (I):

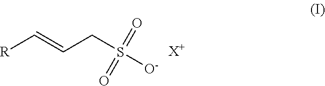

wherein:
   R is selected from H or alkyl chain that has 1-24 carbon atoms, preferably 6-24 carbon atoms, more preferably, 8 to 20 carbon atoms, said chain being saturated or unsaturated, linear or branched, substituted or unsubstituted, and
   M⁺ is chosen from any suitable monovalent cation.

In some embodiments in formula (I), useful cations are alkali metal ions such as sodium or potassium, ammonium ions, or alkanolammonium ions such as monoethanolammonium or triethanolammonium ions.

In some instances, the alkyl sulfonate(s) are chosen from linear or branched C1-C30, C2-C28, or C4-C24 alkyl sulfonates, for example C8-C16 alkyl benzene sulfonates, C10-C20 paraffin sulfonates, C10-C24 olefin sulfonates, salts thereof, or mixtures thereof. In some preferred embodiments, C10-C24, C12-C20, or C12-C18 olefin sulfonates and/or salts thereof may be chosen. A non-limiting example of a C10-C24 olefin sulfonate that can be used is sodium C14-16 olefin sulfonate.

Alkyl sulfosuccinates may, for example, be chosen from linear or branched C2-C30, such as C4-C30, C6-C30, or C8-C30 alkyl sulfosuccinates. Non-limiting examples of useful alkyl sulfosuccinates and their salts include those of formula (II):

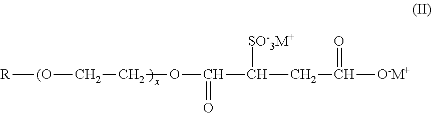

wherein:
   R is a straight or branched chain alkyl or alkenyl group having 10 to 22 carbon atoms, preferably 10 to 20 carbon atoms;
   x is a number that represents the average degree of ethoxylation, and can range from 0 to about 5, preferably from 0 to about 4, and most preferably from about 2 to about 3.5; and
   M, which can be the same or different, is chosen from any suitable monovalent cation.

In some embodiments in formula (II), useful cations are alkali metal ions such as sodium or potassium, ammonium ions, or alkanolammonium ions such as monoethanolammonium or triethanolammonium ions.

Non-limiting examples of alkyl sulfosuccinate salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or mixtures thereof.

Alkyl sulfoacetates may, for example, be chosen from linear or branched C2-C30, such as C4-C30, C6-C30, or C8-C30 alkyl sulfoacetates. Non-limiting examples of alkyl sulfoacetates and their salts include C4-C18 fatty alcohol sulfoacetates and/or salts thereof. In some embodiments, a sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

Non-limiting examples of useful acyl isethionates and their salts include those of formula (III):

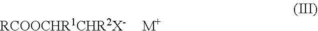

wherein:
   R, R¹, and R² are each independently chosen from H or an alkyl chain having 1-24 carbon atoms, said chain being saturated or unsaturated, linear or branched;
   X is SO₃⁻; and
   M is any suitable cation.

Although the cation in formula (III) may be chosen from any suitable cation including, for example, alkali metal ions such as sodium or potassium, ammonium ions, or alkanolammonium ions such as monoethanolammonium or triethanolammonium ions, sodium is a preferred cation.

In various embodiments, RCO— represents the coconut acid moiety. Non-limiting examples of acyl isethionates include sodium cocoyl isethionate, sodium lauroyl isethionate, sodium lauroyl methyl isethionate, and sodium cocoyl methyl isethionate.

Other useful non-sulfate anionic surfactants include, for example, alkoxylated monoacids, and acyl amino acids such as acyl taurates, acyl glycinates, acyl glutamates, acyl sarcosinates, as well as salts thereof and mixtures thereof.

Non-limiting examples of alkoxylated monoacids include compounds corresponding to formula (IV):

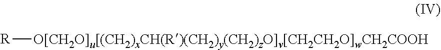

wherein:
   R is a hydrocarbon radical containing from about 6 to about 40 carbon atoms;
   R′ represents hydrogen or alkyl;
   u, v, and w, which may be identical or different, independently represent numbers from 0 to 60;
   x, y, and z, which may be identical or different, independently represent numbers from 0 to 13; and
   the sum of x+y+z>0.

Compounds corresponding to formula (IV) can be obtained by alkoxylation of alcohols R—OH with ethylene oxide as the sole alkoxide or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (IV), R is linear or branched, acyclic or cyclic, saturated or unsaturated, aliphatic or aromatic, substituted or unsubstituted. For example, R may be a linear or branched, acyclic C6-C40 alkyl or alkenyl group or a C1-C40 alkyl phenyl group, more typically a C8-C22 alkyl or alkenyl group, or a C4-C18 alkyl phenyl group, and even more typically a C12-C18 alkyl group or alkenyl group or a C6-C16 alkyl phenyl group. Further, u, v, w, independently of one another, may be chosen from a number ranging from 2 to 20, such as a number ranging from 3 to 17, or a number ranging from 5 to 15. Further still, x, y, z, independently of one another, may be chosen from a number ranging from 0 to 13, such as a number ranging from 1 to 10, or a number ranging from 2 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, PPG-5-Ceteth-20, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid, or mixtures thereof. In some cases, preferred ethoxylated acids include Oleth-10 Carboxylic Acid, Laureth-11 Carboxylic Acid, or mixtures thereof.

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. A useful cation associated with the acyl amino acid can be sodium or potassium. Alternatively, the cation can be an organic salt such as triethanolamine (TEA) or a metal salt.

Non-limiting examples of acyl amino acids include those of formula (V):

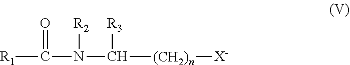

wherein:
   R¹, R², and R³ are each independently selected from H or an alkyl chain having 1-24 carbon atoms, said chain being saturated or unsaturated, linear or branched, substituted or unsubstituted;
n ranges from 0 to 30; and
X is COO⁻ or SO₃⁻.

Non-limiting examples of acyl taurates include those of formula (VI):

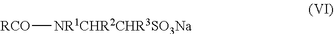

wherein R, R¹, R², and R³ are each independently selected from H or an alkyl chain having from 1-24 carbon atoms, such as from 6-20 carbon atoms, or from 8-16 carbon atoms, said chain being saturated or unsaturated, linear or branched, substituted or unsubstituted.

In various embodiments, RCO— represents the coconut acid moiety. Non-limiting examples of acyl taurate salts include sodium cocoyl taurate and sodium methyl cocoyl taurate.

Non-limiting examples of useful acyl glycinates include those of formula (VII):

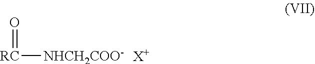

wherein:
   R is an alkyl chain of 8 to 16 carbon atoms, and
   X⁺ is chosen from any suitable monovalent cation.

In some embodiments in formula (VII), useful cations are alkali metal ions such as sodium or potassium, ammonium ions, or alkanolammonium ions such as monoethanolammonium or triethanolammonium ions.

Non-limiting examples of acyl glycinates include sodium cocoyl glycinate, sodium lauroyl glycinate, sodium myristoyl glycinate, potassium lauroyl glycinate, and potassium cocoyl glycinate.

Non-limiting examples of useful acyl glutamates include those of formula (VIII):

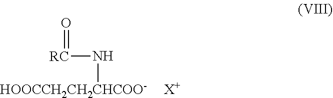

wherein:
   R is an alkyl chain of 8 to 16 carbon atoms, and
   X⁺ is chosen from any suitable monovalent cation.

In some embodiments in formula (VIII), useful cations are alkali metal ions such as sodium or potassium, ammonium ions, or alkanolammonium ions such as monoethanolammonium or triethanolammonium ions.

Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate.

Non-limiting examples of acyl sarcosinates and their salts include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

In various embodiments, the second anionic surfactant chosen from sulfo-derived surfactants is chosen from olefin sulfonates and/or salts thereof. In a preferred embodiment, the second anionic surfactant is chosen from C10-C24, C12-C20, or C12-C18 olefin sulfonates and/or salts thereof, for example sodium C14-16 olefin sulfonate.

In further embodiments, the second anionic surfactant comprises at least one surfactant chosen from acyl taurates, acyl glycinates, acyl glutamates, acyl sarcosinates, salts thereof, or mixtures thereof, particularly acyl sarcosinates. In still further embodiments, the second anionic surfactant comprises at least one sulfo-derived surfactant chosen from alkyl sulfonates, salts thereof, or mixtures thereof, and optionally at least one surfactant chosen from acyl taurates, acyl glycinates, acyl glutamates, acyl sarcosinates, salts thereof, or mixtures thereof.

As an exemplary and non-limiting embodiment, a surfactant system may comprise at least one first anionic surfactant chosen from sulfosuccinates, e.g. C8-C30 alkyl sulfosuccinates, sulfoacetates, e.g. C8-C30 alkyl sulfoacetates, acyl sarcosinates, salts thereof, or combinations of two or more thereof, and optionally at least one second anionic surfactant chosen from sulfo-derived surfactants such as sulfosuccinates, sulfoacetates, isethionates, and alkyl sulfonates, e.g. C4-C28 alkyl sulfonates.

As a further exemplary and non-limiting embodiment, a surfactant system may comprise at least one first anionic surfactant chosen from C8-C30 alkyl sulfosuccinates, C8-C30 alkyl sulfoacetates, acyl sarcosinates, salts thereof, or combinations of two or more thereof, and at optionally least one second anionic surfactant chosen from C10-C24, C12-C20, or C12-C18 olefin sulfonates and/or salts thereof, for example sodium C14-16 olefin sulfonate.

As yet a further exemplary and non-limiting embodiment, a surfactant system may comprise at least one first anionic surfactant chosen from disodium laureth sulfosuccinate, sodium lauryl sulfoacetate, sodium lauroyl sarcosinate, or combinations of two or more thereof, and optionally at least one second anionic surfactant. For example, the surfactant system may comprise at least one first anionic surfactant chosen from disodium laureth sulfosuccinate, sodium lauryl sulfoacetate, sodium lauroyl sarcosinate, or combinations of two or more thereof, and at least one second anionic surfactant chosen from C10-C24, C12-C20, or C12-C18 olefin sulfonates and/or salts thereof, for example sodium C14-16 olefin sulfonate.

The total amount of anionic surfactants may vary. For example, the total amount of anionic surfactants may range from about 1% to about 20%, such as from about 1.5% to about 18%, from about 2% to about 15%, from about 2.5% to about 12%, or from about 3% to about 10% by weight, including ranges and sub-ranges there between, relative to the total weight of the composition. In various embodiments, the total amount of anionic surfactants may range from about 1% to about 20%, from about 1% to about 18%, from about 1% to about 15%, from about 1% to about 12%, from about 1% to about 10%, from about 1% to about 9%, from about 1% to about 8%, from about 2% to about 20%, from about 2% to about 18%, from about 2% to about 15%, from about 2% to about 12%, from about 2% to about 10%, from about 2% to about 9%, from about 2% to about 8%, from about 3% to about 20%, from about 3% to about 18%, from about 3% to about 15%, from about 3% to about 12%, from about 3% to about 10%, from about 3% to about 9%, from about 3% to about 8%, from about 4% to about 20%, from about 4% to about 18%, from about 4% to about 15%, from about 4% to about 12%, from about 4% to about 10%, from about 4% to about 9%, from about 4% to about 8%, from about 5% to about 20%, from about 5% to about 18%, from about 5% to about 15%, from about 5% to about 12%, from about 5% to about 10%, from about 5% to about 9%, from about 5% to about 8%, from about 6% to about 20%, from about 6% to about 18%, from about 6% to about 15%, from about 6% to about 12%, from about 6% to about 10%, from about 6% to about 9%, from about 6% to about 8%, from about 7% to about 20%, from about 7% to about 18%, from about 7% to about 15%, from about 7% to about 12%, from about 7% to about 10%, from about 7% to about 9%, or from about 7% to about 8% by weight, based on the total weight of the composition.

The total amount of first and second anionic surfactants may also vary. For example, the total amount of first and/or second anionic surfactants may independently range from about 0.5% to about 15%, such as from about 0.75% to about 12%, from about 1% to about 10%, from about 1.5% to about 8%, or from about 2% to about 7% by weight, including ranges and sub-ranges there between, relative to the total weight of the composition. In various embodiments, the total amount of first and/or second anionic surfactants may independently range from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 3% to about 4%, from about 4% to about 12%, from about 4% to about 11%, from about 4% to about 10%, from about 4% to about 9%, from about 4% to about 8%, from about 4% to about 7%, from about 4% to about 6%, or from about 4% to about 5% by weight, based on the total weight of the composition.

### Amphoteric Surfactants

Compositions according to the disclosure optionally comprise at least one amphoteric surfactant. It is to be understood that salts of amphoteric surfactants are expressly included, whether or not stated.

Non-limiting examples of amphoteric surfactants that can be used include betaines, alkyl sultaines, alkyl amphoacetates, amphopropionates, or mixtures thereof.

In various embodiments, non-limiting examples of betaines or salts thereof the at least one amphoteric surfactant may comprise alkyl betaines, amido betaines, or mixtures thereof. In various embodiments, the compositions comprise at least one compound chosen from (C₈-C₂₀) alkylbetaines, sulfobetaines, (C₈-C₂₀) alkylamido (C₆-C₈) alkylbetaines, (C₈-C₂₀) alkylamido (C₆-C₈) alkylsulfobetaines, salts thereof, or mixtures thereof.

In some embodiments, exemplary useful betaines include, but are not limited to, those of the following formulae (IX)-(XII):

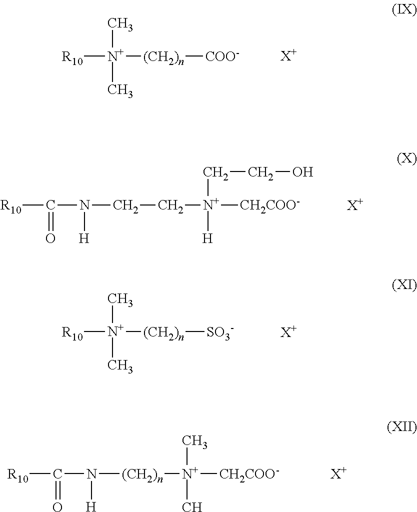

wherein:
   R₁₀ is an alkyl group having from 8 to 18 carbon atoms;
   n is an integer ranging from 1 to 3; and
   X⁺ is a cationic counterion.

Useful betaines include, for example, cocobetaine, cocamidopropyl betaine, cetyl betaine, lauryl betaine, laurylhydroxy sulfobetaine, lauryldimethyl betaine, behenyl betaine, capryl/capramidopropyl betaine, stearyl betaine, salts thereof, or mixtures thereof. Cocobetaine is particularly preferred. In some embodiments, however, the composition is free or substantially free of cocamidopropyl betaine.

Non-limiting examples of useful alkyl sultaines include linear or branched C4-C28, C8-C20, or C8-C18 alkyl sultaines. For example, alkyl sultaines may be chosen from those having the following formula (XIII):

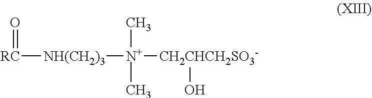

wherein R is an alkyl group having from 8 to 18 carbon atoms.

Examples of alkyl sultaines that can be chosen include cocamidopropyl hydroxysultaine and lauryl hydroxysultaine.

Non-limiting examples of useful alkyl amphoacetates include linear or branched C4-C28, C8-C20, or C8-C18 alkyl amphoacetates. Useful alkyl amphoacetates include, for example, those having the formula (XIV):

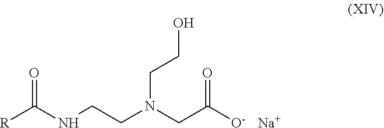

wherein R is an alkyl group having from 8 to 18 carbon atoms.

Non-limiting examples of alkyl amphoacetates include sodium cocoamphoacetate, sodium lauroamphoacetate, sodium caproamphoacetate, and sodium capryloamphoacetate.

Non-limiting examples of useful alkyl amphodiacetates include linear or branched C4-C28, C8-C20, or C8-C18 alkyl amphodiacetates. Useful alkyl amphodiacetates include, for example, those having the formula (XV):

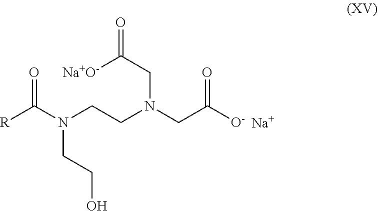

wherein R is an alkyl group having from 8 to 18 carbon atoms.

Nonlimiting examples of useful alkyl amphodiacetates include disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid, and cocoamphodipropionic acid.

The amphoteric surfactants of the present disclosure may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Among the optionally quaternized secondary or tertiary aliphatic amine derivatives that may be used, mention may also be made of the products of respective structures (A1) and (A2) below:

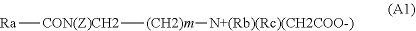

wherein:
   Ra represents a C10-C30 alkyl or alkenyl group derived from an acid Ra—COOH preferably present in hydrolysed coconut oil, a heptyl group, a nonyl group or an undecyl group,
   Rb represents a β-hydroxyethyl group,
   Rc represents a carboxymethyl group;
   m is equal to 0, 1 or 2, and
   Z represents a hydrogen atom or a hydroxyethyl or carboxymethyl group;

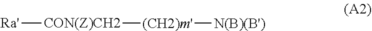

wherein:
   B represents —CH2CH2OX′, with X′ representing —CH2-COOH, CH2-COOZ′, CH2CH2-COOH, —CH2CH2-COOZ′, or a hydrogen atom,
   B′ represents —(CH2)z-Y′, with z=1 or 2, and Y′ representing COOH, COOZ′, CH2-CHOH—SO3H or —CH2-CHOH—SO3Z′,
   m′ is equal to 0, 1 or 2,
   Z represents a hydrogen atom or a hydroxyethyl or carboxymethyl group,
   Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanol-amine, diisopropanolamine or triisopropanolamine, 2-amino-2-methyl-1-propanol, 2-amino-2-methyl-1,3-propanediol and tris(hydroxymethyl)aminomethane, and
   Ra′ represents a C10-C30 alkyl or alkenyl group of an acid Ra′COOH preferably pre-sent in hydrolysed linseed oil or coconut oil, an alkyl group, in particular a C17 alkyl group, and its iso form, or an unsaturated C17 group.

Exemplary amphoteric surfactants include sodium cocoamphoacetate, sodium lauroamphoacetate, sodium caproamphoacetate and sodium capryloamphoacetate. Further exemplary amphoteric surfactants include disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid, and cocoamphodipropionic acid.

Non-limiting examples that may be mentioned include the cocoamphodiacetate sold by the company Rhodia under the trade name Miranol® C2M Concentrate, the sodium cocoamphoacetate sold under the trade name Miranol Ultra C 32, or the product sold by the company Chimex under the trade name CHIMEXANE HA.

Use may also be made of compounds of formula (XVI):

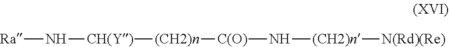

wherein:
   Ra″ represents a C10-C30 alkyl or alkenyl group of an acid
   Ra″—C(O)OH preferably present in hydrolysed linseed oil or coconut oil;
   Y″ represents the group —C(O)OH, —C(O)OZ″, —CH2-CH(OH)—SO3H or the group CH2-CH(OH)—SO3-Z″, with Z″ representing a cationic counterion resulting from an alkali metal or alkaline-earth metal, such as sodium, an ammonium ion or an ion resulting from an organic amine;
   Rd and Re represent, independently of each other, a C1-C4 alkyl or hydroxyalkyl radical; and
   n and n′ denote, independently of each other, an integer ranging from 1 to 3.

In some embodiments, the compositions comprise at least one amphoteric surfactant chosen from betaines, alkyl sultaines, alkyl amphodiacetates, salts thereof, or mixtures thereof. By way of non-limiting example, in some embodiments the compositions include one or more amphoteric surfactants chosen from, cocobetaine, cocamidopropyl betaine, cetyl betaine, lauryl betaine, laurylhydroxy sulfobetaine, lauryldimethyl betaine, stearyl betaine, cocoamphodiacetate, behenyl betaine, capryl/capramidopropyl betaine, lauryl hydroxysultaine, cocamidopropyl hydroxysultaine, cocoamphodiacetate, salts thereof, or mixtures thereof. In some embodiments, however, the composition is free or substantially free of cocamidopropyl betaine.

In preferred embodiments, compositions according to the disclosure comprise at least one amphoteric surfactant chosen from betaines, alkyl hydroxysultaines, alkyl amphodiacetates, salts thereof, or combinations thereof. For example, the amphoteric surfactant(s) may be chosen from cocobetaine, cocamidopropyl hydroxysultaine, and/or disodium cocoamphodiacetate.

If present, the total amount of amphoteric surfactants may vary, but may, for example range from about 0.5% to about 10%, preferably from about 1% to about 8%, more preferably from about 1.5% to about 6%, most preferably from about 2% to about 5.5% by weight, relative to the total weight of the composition. For example, the amphoteric surfactant may be present in the composition in an amount ranging from about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 8%, about 1% to about 7%, about 1% to about 6%, about 1% to about 5%, about 1% to about 4%, about 1% to about 3%, about 1% to about 2%, about 1.5% to about 8%, about 1.5% to about 7%, about 1.5% to about 6%, about 1.5% to about 5%, about 1.5% to about 4%, about 1.5% to about 3%, about 1.5% to about 2%, about 2% to about 8%, about 2% to about 7%, about 2% to about 6%, about 2% to about 5%, about 2% to about 4%, about 2% to about 3%, about 2.5% to about 8%, about 2.5% to about 7%, about 2.5% to about 6%, about 2.5% to about 5%, about 2.5% to about 4%, about 2.5% to about 3%, about 3% to about 8%, about 3% to about 7%, about 3% to about 6%, about 3% to about 5%, about 3% to about 4%, about 4% to about 8%, about 4% to about 7%, about 4% to about 6%, about 4% to about 5%, about 5% to about 8%, about 5% to about 7%, or about 5% to about 6% by weight, relative to the total weight of the composition. By way of example, the amphoteric surfactants may be present in a total amount of about 2%, about 2.1%, about 2.2%, about 2.3%, about 2.4%, about 2.5%, about 2.6%, about 2.7%, about 2.8%, about 2.9%, or about 3%, including all ranges and subranges using any of the foregoing as upper and lower limits. As a further example, the amphoteric surfactants may be present in a total amount of about 5%, about 5.1%, about 5.2%, about 5.3%, about 5.4%, about 5.5%, about 5.6%, about 5.7%, about 5.8%, about 5.9%, or about 6%, including all ranges and subranges using any of the foregoing as upper and lower limits.

It has been discovered that by choosing not only the types and amounts of anionic and amphoteric surfactants, but also the amounts relative to each other, the properties of compositions described herein are surprisingly improved, for example the ability of the compositions to form a foam and/or to deposit beneficial components such as ceramides onto the hair. Therefore, in some embodiments where the compositions comprise amphoteric surfactants, it may be particularly advantageous to choose amounts of anionic surfactants and amphoteric surfactants to achieve a weight ratio of the total amount of anionic surfactants to the total amount of amphoteric surfactants that is less than about 10:1, more particularly less than about 9:1, less than about 8:1, less than about 7:1, less than about 6:1, less than about 5:1, less than about 4:1, less than about 3.5:1, less than about 3:1, less than about 2.5:1, or less than about 2:1.

For example, in some embodiments, the weight ratio of the total amount of anionic surfactants to the total amount of amphoteric surfactants ranges from about 1:1 to about 10:1, such as from about 1:1 to about 9:1, from about 1:1 to about 8:1, from about 1:1 to about 7:1, from about 1:1 to about 6:1, from about 1:1 to about 5:1, from about 1:1 to about 4:1, from about 1:1 to about 3.5:1, from about 1:1 to about 3:1, from about 1:1 to about 2.5:1, from about 1:1 to about 2:1, from about 1.5:1 to about 9:1, from about 1.5:1 to about 8:1, from about 1.5:1 to about 7:1, from about 1.5:1 to about 6:1, from about 1.5:1 to about 5:1, from about 1.5:1 to about 4:1, from about 1.5:1 to about 3.5:1, from about 1.5:1 to about 3:1, from about 1.5:1 to about 2.5:1, from about 1.5:1 to about 2:1, from about 2:1 to about 9:1, from about 2:1 to about 8:1, from about 2:1 to about 7:1, from about 2:1 to about 6:1, from about 2:1 to about 5:1, from about 2:1 to about 4:1, from about 2:1 to about 3.5:1, from about 2:1 to about 3:1, from about 2:1 to about 2.5:1, from about 2.5:1 to about 9:1, from about 2.5:1 to about 8:1, from about 2.5:1 to about 7:1, from about 2.5:1 to about 6:1, from about 2.5:1 to about 5:1, from about 2.5:1 to about 4:1, from about 2.5:1 to about 3.5:1, from about 2.5:1 to about 3:1, from about 3:1 to about 9:1, from about 3:1 to about 8:1, from about 3:1 to about 7:1, from about 3:1 to about 6:1, from about 3:1 to about 5:1, from about 3:1 to about 4:1, or from about 3:1 to about 3.5:1, or is about 1.5:1, about 1.6:1, abut 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.9:1, about 4:1, about 4.1:1, about 4.2:1, about 4.3:1, about 4.4:1, or about 4.5:1, including all ranges and subranges using any of the foregoing as upper and lower limits.

### Nonionic Surfactants

In various embodiments, the compositions may further comprise at least one nonionic surfactant. However, in certain embodiments, the compositions are free or essentially free of nonionic surfactants. Salts of nonionic surfactants are expressly included, whether or not stated. If present, useful nonionic surfactants can include, for example, alkyl polyglucosides, glycol ethers, amine oxides, or mixtures thereof.

In certain embodiments, the nonionic surfactant may be chosen from fatty acid amides, alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof.

Useful fatty acid amides may be chosen from compounds derived from amides of alkanolamines and amides of saturated or unsaturated linear or branched C8-C30 fatty acids, the alkanolamines and/or fatty acids being optionally oxyalkylenated, more particularly oxyethylenated, with 1 to 50 mol of ethylene oxide. Optionally, useful fatty acid amides can be chosen from C2-C10 alkanolamines and amides of C14-C30 fatty acids, for example C2-C10 alkanolamines and amides of C14-C22 fatty acids.

In some embodiments, the fatty acid amide is coconut fatty acid monoisopropanolamide, such as cocamide MIPA oleic diethanolamide, myristic acid monoethanolamide, soy fatty acid diethanolamide, stearic acid ethanolamide, oleic acid monoisopropanolamide, linoleic acid diethanolamide, It can be selected from stearic acid monoethanolamide, behenic acid monoethanolamide, isostearic acid monoisopropanolamide, erucic acid diethanolamide, ricinoleic acid monoethanolamide, and canola seed fatty acid amides containing 4 mol of ethylene oxide. Most preferably, fatty acid amides include cocamide MIPA.

Exemplary alkyl and polyalkyl esters of poly(ethylene oxide) include those containing at least one C8-C30 alkyl radical, with a number of ethylene oxide (EO) units ranging from 2 to 200. Mention may be made, for example, of PEG-20 stearate, PEG-40 stearate, PEG-100 stearate, PEG-20 laurate, PEG-8 laurate, PEG-40 laurate, PEG-55 propylene glycol oleate, PEG-150 distearate, PEG-150 pentaerythrityl tetrastearate, PEG-7 cocoate, PEG-9 cocoate, PEG-8 oleate, PEG-10 oleate, and PEG-40 hydrogenated castor oil.

Useful alkyl and polyalkyl ethers of poly(ethylene oxide) include those containing at least one C8-C30 alkyl radical, with a number of ethylene oxide (EO) units ranging from 3 to 200. Mention may be made, for example, of laureth-3, laureth-4, laureth-5, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and mixtures thereof.

Exemplary and nonlimiting polyoxyethylenated alkyl and polyalkyl esters of sorbitan include those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).

Exemplary polyoxyethylenated alkyl and polyalkyl ethers of sorbitan include those with a number of ethylene oxide (EO) units ranging from 0 to 100.

Exemplary and nonlimiting alkyl and polyalkyl esters of sucrose that may be mentioned are Crodesta™ F150, sucrose monolaurate sold under the name Crodesta SL 40, and the products sold by Ryoto Sugar Ester, for instance sucrose palmitate sold under the reference Ryoto™ Sugar Ester P1670, Ryoto™ Sugar Ester LWA 1695 or Ryoto Sugar™ Ester 01570. Sucrose monooleate, monomyristate and monostearate are also suitable for use.

Exemplary and nonlimiting (poly)oxyethylenated alkyl and polyalkyl esters of glycerol include those with a number of ethylene oxide (EO) units ranging from 0 to 100 and a number of glycerol units ranging from 1 to 30. Mention may be made, for example, of hexaglyceryl monolaurate, PEG-55 propylene glycol oleate, PEG-30 glyceryl stearate, polyglyceryl-2 laurate, polyglyceryl-10 laurate, polyglyceryl-10 stearate, polyglyceryl-10 oleate, PEG-7 glyceryl cocoate and PEG-20 glyceryl isostearate.

Useful examples of (poly)oxyethylenated alkyl and polyalkyl ethers of glycerol include those with a number of ethylene oxide (EO) units ranging from 0 to 100 and a number of glycerol units ranging from 1 to 30. Examples that may be mentioned include Nikkol Batyl Alcohol 100 and Nikkol Chimyl Alcohol 100.

When present, the total amount of nonionic surfactants may vary. For example, in various embodiments compositions according to the disclosure may comprise a total amount of nonionic surfactants ranging from about 0.01% to about 5%, from about 0.05% to about 4%, from about 0.1% to about 3% by weight, from about 0.25% to about 2%, or from about 0.5% to about 1% by weight, relative to the total weight of the composition. In at least some embodiments, the compositions comprise less than about 2%, less than about 1.75%, less than about 1.5%, less than about 1.25%, less than about 1%, or less than about 0.75% of nonionic surfactants.

### Cationic Surfactants

In various embodiments, the compositions may further comprise at least one cationic surfactant. However, in certain embodiments, the compositions are free or essentially free of cationic surfactants. Salts of cationic surfactants are expressly included, whether or not stated. Exemplary and non-limiting cationic surfactants include cationic amine-based or quaternary ammonium-based compounds.

For example, cationic surfactants may be chosen from alkylpyridinium salts, ammonium salts of imidazoline, diquaternary ammonium salts, and ammonium salts containing at least one ester function. As a further example, cationic surfactants may be chosen from quaternary ammonium salts having the following formula (XVII):

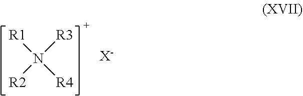

wherein R1 to R4, which may be identical or different, represent a linear or branched aliphatic radical containing from 1 to 30 carbon atoms, or an aromatic radical such as aryl or alkylaryl; the aliphatic radicals may optionally comprise heteroatoms (O, N, S or halogens) and may optionally be substituted, and X⁻ is an anion chosen from the group of halides, phosphates, acetates, lactates, C2-C6 alkyl sulfates and alkyl or alkylarylsulfonates. The aliphatic radicals are chosen, for example, from C12-C22 alkyl, alkoxy, C2-C6 polyoxyalkylene, alkylamide, (C12-C22)alkylamido(C2-C6)alkyl, (C12-C22)alkyl-acetate and hydroxyalkyl radicals, containing from 1 to 30 carbon atoms.

As a further example, quaternary ammonium salts containing at least one ester function, such as those of formula (XVIII) may be chosen:

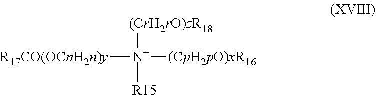

wherein:
   R15 is chosen from C1-C6 alkyl radicals and C1-C6 hydroxyalkyl or dihydroxyalkyl radicals;
   R16 is chosen from the radical R19-CO—, linear or branched, saturated or unsaturated C1-C22 hydrocarbon-based radicals R20, a hydrogen atom;
   R18 is chosen from the radical R21-CO, linear or branched, saturated or unsaturated C1-C22 hydrocarbon-based radicals R22, a hydrogen atom;
   R17, R19 and R21, which may be identical or different, are chosen from linear or branched, saturated or unsaturated C7-C21 hydrocarbon-based radicals;
   r, n, and p, which may be identical or different, are integers ranging from 2 to 6;
   y is an integer ranging from 1 to 10;
   x and z, which may be identical or different, are integers ranging from 0 to 10; and
   X⁻ is a simple or complex organic or mineral anion;
with the provisos that:
   the sum x+y+z is from 1 to 15,
   when x is 0, then R16 denotes R20, and
   when z is 0, then R18 denotes R22.

In formula (XVIII), the alkyl radicals R15 may be linear or branched, and more particularly linear. Preferably, R15 denotes a methyl, ethyl, hydroxyethyl or dihydroxypropyl radical, and more particularly a methyl or ethyl radical. Advantageously, the sum x+y+z is from 1 to 10. When R16 is a hydrocarbon-based radical R20, it may contain from 12 to 22 carbon atoms, or contain from 1 to 3 carbon atoms. When R18 is a hydrocarbon-based radical R22, it preferably contains 1 to 3 carbon atoms. Advantageously, R17, R19, and R21, which may be identical or different, are chosen from linear or branched, saturated or unsaturated C11-C21 hydrocarbon-based radicals, and more particularly from linear or branched, saturated or unsaturated C11-C21 alkyl and alkenyl radicals. Preferably, x and z, which may be identical or different, are equal to 0 or 1. Advantageously, y is equal to 1. Preferably, r, n and p, which may be identical or different, are equal to 2 or 3 and even more particularly equal to 2. The anion X⁻ is preferably a halide (chloride, bromide or iodide) or a C1-C4 alkyl sulfate, more particularly methyl sulfate. The anion X⁻ may also represent methanesulfonate, phosphate, nitrate, tosylate, an anion derived from an organic acid (such as acetate or lactate), or any other anion that is compatible with the ammonium containing an ester function.

The surfactants may be, for example, the salts (chloride or methyl sulfate) of diacyloxyethyldimethylammonium, of diacyloxyethylhydroxyethyldimethylammonium, of monoacyloxyethylhydroxyethyldimethylammonium, of triacyloxyethylmethylammonium, of monoacyloxyethylhydroxyethyldimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

Other suitable cationic surfactants are esterquats which are quaternary ammonium compounds having fatty acid chains containing ester linkages, such as, for example, dibehenoylethyl dimonium chloride, dipalmitoylethyl dimonium chloride, distearoylethyl dimonium chloride, ditallowoyl PG-dimonium chloride, dipalmitoylethyl hydroxyethylmonium methosulfate, distearoylethyl hydroxyethylmonium methosulfate, or mixtures thereof.

If present, the compositions comprise cationic surfactants in an amount ranging from about 0.001% to about 5%, from about 0.01% to about 2.5%, from about 0.025% to about 2%, from about 0.05% to about 1%, or from about 0.05% to about 0.5% by weight, relative to the total weight of the composition. In at least some embodiments, the compositions comprise less than about 0.5%, less than about 0.25%, less than about 0.1%, less than about 0.09%, less than about 0.08%, or less than about 0.07% of cationic surfactants.

### Scalp Benefit Agents

Compositions according to the disclosure comprise at least one scalp benefit agent. Scalp benefit agents provide benefits to the scalp such as improved skin barrier functionality, or promoting regeneration, renewal, repair and/or improved moisture retention of the scalp. Non-limiting examples of scalp benefit agents that may be chosen include sterols such as cholesterol and lanosterol, vitamins and analogues thereof such as B1 (thiamine), B3 (niacinamide), B5 (pantothenic acid), and E (tocopherol acetate), phytosphingosine, ceramides such as ceramide AP, ceramide NP, ceramide EOP, and combinations of two or more thereof. Preferably, scalp benefit agents that may be chosen include ceramide AP, ceramide NP, ceramide EOP, or combinations of two or more thereof.

Typically, the total amount of scalp benefit agents present in the composition is less than 1%, such as less than 0.5%, less than 0.25%, less than 0.1%, or less than 0.05% by weight, relative to the total weight of the composition. For example, in various embodiments, the total amount of scalp benefit agents ranges from about 0.0001% to about 1%, such as from about 0.0001% to about 0.5%, from about 0.0001% to about 0.25%, from about 0.0001% to about 0.1%, from about 0.0001% to about 0.05%, from about 0.0001% to about 0.01%, from about 0.0005% to about 1%, from about 0.0005% to about 0.5%, from about 0.0005% to about 0.25%, from about 0.0005% to about 0.1%, from about 0.0005% to about 0.05%, from about 0.0005% to about 0.01%, from about 0.001% to about 1%, from about 0.001% to about 0.5%, from about 0.001% to about 0.25%, from about 0.001% to about 0.1%, from about 0.001% to about 0.05%, or from about 0.001% to about 0.01% by weight, relative to the total weight of the composition. In certain embodiments, the compositions comprise a total amount of scalp benefit agents chosen from ceramide AP, ceramide NP, ceramide EOP, and combinations of two or more thereof ranging from about 0.0001% to about 1%, such as from about 0.0001% to about 0.5%, from about 0.0001% to about 0.25%, from about 0.0001% to about 0.1%, from about 0.0001% to about 0.05%, from about 0.0001% to about 0.01%, from about 0.0005% to about 1%, from about 0.0005% to about 0.5%, from about 0.0005% to about 0.25%, from about 0.0005% to about 0.1%, from about 0.0005% to about 0.05%, from about 0.0005% to about 0.01%, from about 0.001% to about 1%, from about 0.001% to about 0.5%, from about 0.001% to about 0.25%, from about 0.001% to about 0.1%, from about 0.001% to about 0.05%, or from about 0.001% to about 0.01% by weight, relative to the total weight of the composition.

### Conditioning Agents

It may be advantageous to include one or more hair conditioning agents in compositions according to the disclosure. If present, conditioning agents may be chosen from, for example, cationic conditioning agents, silicone compounds, non-silicone fatty compounds, or combinations thereof.

Cationic conditioning agents include at least one cationic group and/or at least one group that may be ionized into a cationic group. For example, cationic conditioning agents that can be used may be cationic polymers having at least one amine group chosen from primary, secondary, tertiary, and quaternary amine groups, and such groups may either form part of the main polymer chain or may be borne by a side substituent that is directly attached to the main polymer chain. In various embodiments, cationic conditioning agents may be chosen from amidoamines, monoalkyl quaternary amines, dialkyl quaternary amines, polyquaternium compounds, or salts thereof.

Examples of amidoamines include, but are not limited to, oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, palmitamidopropyl dimethylamine, stearamidoethyldiethylamine, and mixtures thereof.

Non-limiting examples of polyquaternium compounds include polyquaternium 5, polyquaternium 6, polyquaternium 7, polyquaternium 10, polyquaternium 11, polyquaternium 15, polyquaternium 16, polyquaternium 22, polyquaternium 28, polyquaternium 39, polyquaternium 44, polyquaternium 46, polyquaternium 47, and mixtures thereof.

Useful and non-limiting silicone conditioning agents that can be included in compositions according to the disclosure include, but are not limited to, polyorganosiloxanes, polyalkylsiloxanes, polyarylsiloxanes, polyalkarylsiloxanes, polyestersiloxanes, and mixtures thereof. For example, dimethicone, cyclomethicone (cyclopentasiloxane), amodimethicone, trimethyl silyl amodimethicone, phenyl trimethicone, trimethyl siloxy silicate, polymethylsilsesquioxane, or mixtures thereof may be chosen.

In some embodiments, however, the compositions according to the disclosure are free or essentially free of silicones. For example, the compositions may in some embodiments include less than about 3%, less than about 2%, less than about 1%, less than about 0.5%, less than about 0.25%, less than about 0.1%, less than about 0.05%, or less than about 0.01% of silicones.

Useful and non-limiting examples of non-silicone fatty compounds include, for example, lower alkanes, fatty alcohols, esters of fatty acids, esters of fatty alcohols, or combinations thereof. By way of example, non-silicone fatty compounds may be chosen from natural oils, such as coconut oil; hydrocarbons, such as mineral oil and hydrogenated polyisobutene; fatty alcohols, such as octyldodecanol; esters, such as C12-C15 alkyl benzoate; diesters, such as propylene dipelarganate; and triesters, such as glyceryl trioctanoate. Examples of useful low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or mixtures thereof. Examples of useful high viscosity oils include castor oil, lanolin and lanolin derivatives, triisocetyl citrate, sorbitan sesquioleate, C10-C18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, stearyl alcohol, cetyl alcohol, cetearyl alcohol, ceramides, or combinations of two or more thereof.

When present, the total amount of conditioning agents can vary, but will typically be present in a total amount ranging from about 0.001% to about 5%, from about 0.005% to about 4%, from about 0.01% to about 3%, from about 0.025% to about 2%, or from about 0.05% to about 1% by weight, relative to the total weight of the composition.

### Thickening Agents/Rheology Modifiers

The compositions may optionally further comprise at least one thickening agent (also referred to as rheology or viscosity modifying agents). As non-limiting examples, polysaccharide-based thickeners, which are polymers having monosaccharides or disaccharides as base units, may be chosen. The polysaccharide thickeners which can be used in the compositions according to the present invention include, byway of example only, gums, celluloses, starches, or mixtures thereof.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and biosaccharide gum. Modified gums or derivatives of gums may also be used, such as, for example, deacylated gellan gum, welan gum, or hydroxypropylated guar gum, such as Jaguar HP 105 sold by Rhodia.

Non-limiting examples of celluloses include hydroxyalkylcelluloses, such as hydroxyethylcellulose, hydroxypropylmethylcellulose, or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, e.g. nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose.

Non-limiting examples of starches include modified and unmodified starches and starch-based polymers, for example methylhydroxypropyl starch, potato starch, wheat starch, corn starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch. Useful modified starches include hydrolyzed corn starch or modified potato starch.

In various exemplary embodiments, the total amount of thickening agents may vary, but typically ranges from about 0.01% to about 5%, including all ranges and subranges therebetween, such as from about 0.01% to about 4%, from about 0.01% to about 3%, from about 0.01% to about 2%, from about 0.01% to about 1.5%, from about 0.01% to about 1%, from about 0.01% to about 0.5%, from about 0.1% to about 5%, from about 0.1% to about 4%, from about 0.1% to about 3%, from about 0.1% to about 2%, from about 0.1% to about 1.5%, from about 0.1% to about 1%, or from about 0.1% to about 0.5%, by weight, relative to the total weight of the composition. In at least certain embodiments, the composition comprises a thickener in an amount less than about 1%, less than about 0.75%, less than about 0.5%, less than about 0.25%, or less than about 0.1% by weight, relative to the total weight of the composition.

### Solvent(s)

Compositions according to the disclosure comprise at least one solvent. The solvent may be chosen from water, non-aqueous solvents, or mixtures thereof.

In some embodiments, the solvent comprises, consists essentially of, or consists of water. The total amount of water in the compositions may vary depending on the type of composition and the desired consistency, viscosity, etc. In various embodiments, water is present in the compositions in an amount ranging from about 60% to about 95%, such as about 65% to about 90%, about 70% to about 85%, or about 75% to about 85% by weight, relative to the total weight of the composition.

In certain embodiments, the composition comprises one or more non-aqueous solvents, for example, glycerin, C14 alcohols, organic solvents, fatty alcohols, fatty ethers, fatty esters, polyols, glycols, vegetable oils, mineral oils, liposomes, laminar lipid materials, or any a mixture thereof. Non-limiting examples of solvents which may be used include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbitol, sorbitan, acetine, diacetine, triacetine, sulfolane, and a mixture thereof.

The total amount of solvent may range from about 60% to about 98% by weight, relative to the total weight of the composition, including all ranges and subranges therebetween. For example, in one embodiment, the total amount of solvent may be about 70% to about 95%, about 70% to about 90%, about 75% to about 85%, or about 75% to about 80% by weight, relative to the total weight of the composition.

### Auxiliary Components

Compositions according to the disclosure may optionally comprise any auxiliary component suitable for use in such compositions. Such components may include, but are not limited to, suspending agent such as carbomer and/or acrylates for suspending anti-dandruff agents, dyes/pigments, humectants and moisturizing agents, fatty substances, thickeners other than polysaccharide thickeners, fillers, structuring agents, shine agents, antioxidants or reducing agents, penetrants, sequestrants, fragrances, buffers, dispersants, plant extracts, preserving agents, opacifiers, sunscreen agents, pH adjusting agents, and antistatic agents.

Optional auxiliary components may be present in the composition in a total amount ranging up to about 15%, such as up to about 10%, up to about 5%, up to about 3%, up to about 2%, or up to about 1% by weight, relative to the total weight of the composition. For example, compositions according to the disclosure may comprise a total amount of auxiliary components ranging from about 0.001% to about 5%, from about 0.005% to about 4%, from about 0.01% to about 3%, from about 0.05% to about 2.5%, or from about 0.1% to about 2% by weight, relative to the total weight of the composition.

In some embodiments, the compositions comprise at least one anti-dandruff active agent. Useful and non-limiting examples include ellagic acid, ethers of ellagic acid, salts of ellagic acid, salts of ethers of ellagic acid, salts of pyrithione, 1-hydroxy-2-pyridone derivatives, selenium (poly)sulfides, salicylic acid, piroctone olamine, tea tree oil, climbazole, fluocinolone, ketoconazole, coal tar, or combinations of two or more thereof. Zinc pyrithione may be particularly useful. If present, the total amount of anti-dandruff active agents may, for example, range from about 0.01% to about 10%, including all ranges and subranges therebetween, such as from about 0.01% to about 5%, from about 0.01% to about 2.5%, from about 0.1% to about 5%, from about 0.1% to about 2.5%, from about 0.5% to about 5%, from about 0.5% to about 4%, from about 0.5% to about 3%, from about 0.5% to about 2%, from about 0.5% to about 1.5%, from about 0.5% to about 1.25%, from about 0.5% to about 1%, or from about 0.5% to about 0.75% by weight, relative to the total weight of the composition.

The compositions may have a pH less than or equal to 9, such from about 3 to about 8, from about 4 to about 7, or from about 5 to about 6.5.

In various embodiments, the compositions according to the present disclosure are stable, meaning that no phase separation or significant change in pH or viscosity is seen when stored at a temperature ranging from about 4° C. to about 45° C., such as from about 10° C. to about 37° C., or from about 20° C. to about 30° C., for at least about 8 weeks.

In at least some embodiments, compositions according to the disclosure may be mild, display good foaming properties, good detangling and combing properties, good antistatic properties, and/or good stability. The compositions may impart one or more properties such as smoothness, conditioning, excellent detangling, anti-frizz, ease of shaping and/or combing, anti-static, clean and/or smooth appearance, with no weigh-down.

### II. Methods

The present disclosure also relates to methods for cleansing and/or conditioning keratin materials, especially the hair, skin, and/or the scalp, with the compositions disclosed herein. Without limitation, methods of cleansing and/or conditioning keratin materials according to the disclosure may include applying a sufficient amount, or an effective amount, of a composition disclosed herein to a keratin material, such as hair, skin, or scalp, which may be wet, damp, or dry, optionally allowing the composition to remain on the keratin material for a desired amount of time, and optionally rinsing the composition from the keratin material. The composition may optionally be lathered before application to the keratin materials, e.g. in the hands, or may be lathered while on the keratin materials.

In certain embodiments, compositions according to the disclosure are particularly useful for cleansing hair. Additionally, the compositions provide a variety of desirable cosmetic and styling benefits to the hair, for example, smoothness without weight-down, detangling, and anti-frizz. As such, the compositions are useful in methods for cleansing hair, as well as methods for imparting smoothness, detangling, and/or frizz control to hair. Accordingly, the disclosure encompasses methods for cleansing hair and methods for treating hair with the compositions of the instant disclosure.

Such methods typically include applying an effective amount of a composition of the instant disclosure to the hair, allowing the composition to remain on the hair for a period of time, and subsequently rinsing the composition from the hair, followed by allowing the hair to air dry or drying the hair with a hair dryer which blows air through the hair and accelerates drying. Usually, the composition is merely allowed to remain on the hair for a period of time sufficient to incorporate the composition throughout the hair, for example, by lathering the composition throughout the hair using one's hands.

The amount of time is sufficient for the composition to interact with the hair and any dirt, oil, contamination, etc., that may exist on the hair so that when rinsed, the dirt, oil, contamination, etc., can be effectively removed from the hair and the conditioning agents of the composition can interact with the hair to condition it. Thus, the composition may be allowed to remain on the hair for a leave-in period of up to about 30 minutes, such as from about 5 seconds to about 15 minutes, about 5 seconds to about 10 minutes, about 5 seconds to about 5 minutes, about 10 seconds to about 5 minutes, or about 10 seconds to about 3 minutes. Alternatively, the composition may be removed immediately once lathering is complete, e.g. without any leave-in period. The composition is then rinsed from the hair, and the hair allowed to dry.

As is common when using shampoo compositions, the hair may be wetted or rinsed with water prior to application of a composition disclosed herein. Having water already in the hair may be helpful for creating lather when applying the compositions because the water interacts with the surfactants.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

As used herein, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “water and/or non-aqueous solvents” means “water and non-aqueous solvents” as well as “water or non-aqueous solvents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Unless expressly stated otherwise, “about” means +/−5%. Likewise, all endpoints of ranges are understood to be individually disclosed, such that, for example, a range of 1:2 to 2:1 is understood to disclose a ratio of both 1:2 and 2:1.

All amounts and ratios herein are given by weight, based upon the total weight of the composition, unless otherwise indicated. Unless otherwise indicated, all percentages herein are by weight of active material.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

As used herein, the terms “applying a composition onto keratin materials,” “applying a composition onto hair,” and variations of these phrases are intended to mean contacting the keratin material or hair with at least one of the compositions according to the disclosure, in any manner.

“Cosmetically acceptable” means compatible with keratinous materials. For example, “cosmetically acceptable carrier” means a carrier that is compatible with any keratinous tissue.

“Keratin material” as used herein includes skin and hair, such as hair on the human head, or hair comprising of eyelashes or hair on the body.

As used herein, the term “conditioning” means imparting to keratin materials at least one property chosen from combability, moisture, luster, shine, and softness. The state of conditioning can be evaluated by any means known in the art, such as, for example, measuring, and comparing, the ease of combability of the treated hair and of the untreated hair in terms of combing work, and consumer perception.

In the present disclosure, reference to a “shampoo” composition means a composition that is intended for cleansing keratin fibers. Although shampoo compositions may also cleanse skin such as the scalp, those skilled in the art understand that the primary purpose of a shampoo composition is to cleanse keratin fibers such as hair that grows from the scalp.

As used herein, the term “treat” (and its grammatical variations) refers to the application of the compositions of the disclosure onto the surface of keratin materials, such as hair.

As used herein, the term “salts” refers to throughout the disclosure may include salts having a counter-ion such as an alkali metal, alkaline earth metal, or ammonium counterion. This list of counterions, however, is non-limiting.

Unless otherwise defined for any specific embodiment, the term “substantially free” and “essentially free,” which are used interchangeably herein, mean that there is less than about 2% by weight of a specific material added to a composition, based on the total weight of the composition. For example, the compositions may include less than about 1.5%, less than about 1%, less than about 0.5%, less than about 0.1%, less than about 0.01%, less than about 0.001%, or less than about 0.0001% of the specified material. As such, it is contemplated that any component described herein for use in the compositions can be present in the compositions in amounts less than about 2%, less than about 1.5%, less than about 1%, less than about 0.5%, less than about 0.1%, less than about 0.01%, less than about 0.001%, or less than about 0.0001%, and the composition will be considered “substantially free” or “essentially free” of such material. A composition that is “free” of a component is understood to contain none of the specified component. However, it is understood that the terms “free” and “substantially free” refer to the amount of a component added to the composition, without including an amount of the component present in the composition as a minor component in a raw material. For example, a composition that is “free” of waxes may not have wax included as an intended component but may nevertheless contain a pigment that is coated with a wax, as such wax would be considered a minor component of the pigment material and would not be expected to provide benefits to the composition that would be expected by including a wax per se as an intended component.

As used herein, the terms “sulfate-based surfactant” and “sulfate-containing surfactant” refer to surfactant compounds that include a sulfate group in their structure.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

Unless otherwise expressly stated, it is in no way intended that any method set forth herein be construed as requiring that its steps be performed in a specific order. Accordingly, where a method does not expressly recite that a particular order of steps must be followed or it is not otherwise specifically stated that the steps are to be limited to a specific order, it is no way intended that any particular order be inferred.

It should also be understood that the precise numerical values used in the specification and claims form additional embodiments of the disclosure and are intended to include any ranges which may be narrowed to any two end points disclosed within the exemplary ranges and values provided, as well as the specific end points themselves.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples that follow serve to illustrate embodiments of the disclosure without, however, being limiting in nature.

### EXAMPLES

The following examples are intended to be non-limiting and explanatory in nature only. In the Examples, amounts are expressed in percentage by weight (wt %) of active materials, relative to the total weight of the composition.

### Example 1—Sulfate-Free Shampoo Compositions

Shampoo compositions 1A-1G according to the disclosure were prepared as set forth in Table 1 below.

TABLE 1A
Shampoo Compositions

| | SHAMPOO | | | | | | |
|---|---|---|---|---|---|---|---|
| | 1A | 1B | 1C | 1D | 1E | 1F | 1G |
| CERAMIDE AP, | 0.0039 | 0.0039 | 0.0039 | 0.0039 | 0.0039 | 0.0039 | 0.0039 |
| CERAMIDE NP, | | | | | | | |
| AND/OR CERAMIDE | | | | | | | |
| EOP | | | | | | | |
| XANTHAN GUM | 0.0049 | 0.0049 | 0.0049 | 0.00075 | 0.00075 | 0.00075 | 0.0049 |
| CATIONIC | 0.02 | 0.02 | 0.08 | 0.02 | 0.02 | 0.02 | 0.02 |
| SURFACTANT | | | | | | | |
| PEG-6 | | 0.1 | | | | | |
| CAPRYLIC/CAPRIC | | | | | | | |
| GLYCERIDES | | | | | | | |
| PEG-150 | | 0.25 | | | | | |
| PENTAERYTHRITYL | | | | | | | |
| TETRASTEARATE | | | | | | | |
| ZINC PYRITHIONE | 1.02 | 1.02 | 1.02 | | | | 1.02 |
| SODIUM LAUROYL | | 4.34 | | | | | |
| SARCOSINATE | | | | | | | |
| POTATO STARCH | | | | | | 0.731 | |
| MODIFIED | | | | | | | |
| SODIUM LAURYL | 1.05 | | 1.05 | 1.05 | 1.05 | 1.05 | 1.05 |
| SULFOACETATE | | | | | | | |
| ACRYLATES | 0.42 | 0.42 | 0.42 | 0.42 | | | 0.42 |
| COPOLYMER | | | | | | | |
| DISODIUM | 2.7 | | 2.7 | 2.7 | 2.7 | 2.7 | 2.7 |
| LAURETH | | | | | | | |
| SULFOSUCCINATE | | | | | | | |
| PHYTOSPHINGOSINE | 0.0027 | 0.0027 | 0.0027 | 0.0027 | 0.0027 | 0.0027 | 0.0027 |
| AND | | | | | | | |
| CHOLESTEROL | | | | | | | |
| GLYCERIN | | | | | | | 0.4 |
| CETEARYL | | | 0.1875 | | | | |
| ALCOHOL | | | | | | | |
| COCAMIDOPROPYL | | 2.5 | | | | | |
| HYDROXYSULTAINE | | | | | | | |
| COCAMIDE MIPA | 0.7 | | 0.7 | 0.7 | 0.7 | 0.7 | 0.7 |
| COCO-BETAINE | 0.27 | 2.97 | 0.27 | 0.27 | 0.27 | 0.27 | 0.27 |
| SODIUM C14-16 | 4.56 | 4.26 | 4.56 | 4.56 | 4.56 | 4.56 | 4.56 |
| OLEFIN | | | | | | | |
| SULFONATE | | | | | | | |
| AMODIMETHICONE | 1.14 | 1.14 | 1.14 | 1.14 | 1.14 | 1.14 | 1.14 |
| POLYQUATERNIUM- | 0.8 | 0.8 | 0.8 | 0.8 | 0.8 | 0.8 | 0.8 |
| 10 | | | | | | | |
| DISODIUM | 2.52 | | 2.52 | 2.52 | 2.52 | 2.52 | 2.52 |
| COCOAMPHO- | | | | | | | |
| DIACETATE | | | | | | | |
| TRIETHYL CITRATE | 0.0017 | 0.0017 | 0.0017 | 0.0017 | 0.0017 | 0.0017 | 0.0017 |
| TRIDECETH-6 | 0.1 | 0.1 | 0.1 | 0.1 | 0.1 | 0.1 | 0.1 |
| CARBOMER | 0.35 | 0.35 | 0.35 | 0.35 | 0.35 | 0.35 | 0.35 |
| SODIUM | 0.01 | 0.01 | 0.01 | 0.01 | 0.01 | 0.01 | 0.01 |
| HYALURONATE | | | | | | | |
| PPG-5-CETETH-20 | 1.0 | 0.5 | 0.5 | 0.5 | 0.5 | 0.5 | 0.5 |
| GLYCOL | 1.35 | 1.35 | 1.35 | 1.35 | 1.35 | 1.35 | 1.35 |
| DISTEARATE | | | | | | | |
| ADDITIVES* | <3 | <3 | <3 | <3 | <3 | <3 | <3 |
| SOLVENTS (water | QS | QS | QS | QS | QS | QS | QS |
| and non-aqueous | to 100 | to 100 | to 100 | to 100 | to 100 | to 100 | to 100 |
| solvents) | | | | | | | |

*preservatives, pH adjusters, stabilizers, consistency agents, electrolytes, antioxidants, emulsifiers

In order to evaluate the properties of shampoo compositions and hair treated with shampoo compositions according to the disclosure compared to other sulfate-free shampoo compositions, cleansing and conditioning routines using shampoo composition 1E from Table 1A and conditioner composition CND in Table 1B were tested against routines using commercially-available shampoo and conditioner compositions, with ingredients as listed on the packaging shown in Table 1C.

TABLE 1B
Conditioner Composition

| | CONDITIONER |
|---|---|
| | CND |
| CETRIMONIUM CHLORIDE | 0.02 |
| PEG-180 | 2.0 |
| BEHENTRIMONIUM CHLORIDE | 4.0 |
| TRIDECETH-6 | 0.09 |
| BEHENTRIMONIUM METHOSULFATE | 0.06 |
| SODIUM HYALURONATE | 0.01 |
| THICKENING AGENTS | 0.20 |
| CONDITIONING COMPOUNDS (amodimethicone, | 8.1 |
| cetyl esters, ceramides, phytosphingosine, | |
| cholesterol, isopropyl alcohol, cetearyl alcohol) | |
| ADDITIVES (preservatives, vitamins, pH adjusters, | <1 |
| stabilizers, antioxidants, chelators, polymers) | |
| SOLVENT (water and non-aqueous solvents) | QS to 100 |

TABLE 1C
Comparative Shampoo and Conditioner Products

| PRODUCT | INGREDIENTS LISTED ON PACKAGE |
|---|---|
| Comparative | Water (Aqua), Sodium C14-16 Olefin Sulfonate, |
| Shampoo #1 | Cocamidopropyl Hydroxysultaine, Sodium Cocoyl Isethionate, |
| C1-S | PPG-2 Hydroxyethyl Coco/Isostearamide, Argania Spinosa |
| | (Argan) Kernel Oil, Panthenol, Dimethicone, Amodimethicone, |
| | Glycol Stearate, PEG-120 Methyl Glucose Dioleate, Guar |
| | Hydroxypropyltrimonium Chloride, Polyquaternium-6, |
| | Polyquaternium-10, Laureth-4, Laureth-23, Cocamidopropyl |
| | Betaine, Cetrimonium Chloride, Trideceth-12, Citric Acid, |
| | Fragrance (Parfum), Tetrasodium EDTA, Sodium Chloride, |
| | Diazolidinyl Urea, Iodopropynyl Butylcarbamate, Yellow 5 (CI |
| | 19140), Red 40 (CI 16035) |
| Comparative | Water (Aqua), Cetyl Alcohol, Cetearyl Alcohol, |
| Conditioner | Behentrimonium Chloride, Glycerin, Steareth-20, PPG-3 |
| C1-C | Benzyl Ether Myristate, Argania Spinosa Kernel Oil, |
| | Amodimethicone, Polyquaternium-47, Polyquaterium-37, |
| | PPG-1 Trideceth-6, Propylene Glycol Dicaprylate/Dicaprate, |
| | Isopropyl Alcohol, Disodium EDTA, Sodium Hydroxide, Citric |
| | Acid, Sodium Benzoate, Fragrance (Parfum), Yellow 5 (CI |
| | 19140), Red 40 (CI 16035) |
| Comparative | Active Ingredient: Pyrithione Zinc 1% Inactive Ingredients: |
| Shampoo #2 | Water, Sodium Lauryl Sulfate, Sodium Laureth Sulfate, Glycol |
| C2 | Distearate, Sodium Chloride, Zinc Carbonate, Fragrance, |
| | Cocamidopropyl Betaine, Sodium Xylenesulfonate, |
| | Dimethicone, Sodium Benzoate, Guar |
| | Hydroxypropyltrimonium Chloride, Magnesium Carbonate |
| | Hydroxide, Blue 1, Red 33 |

### Example 1-1—Properties of Treated Hair Compared to Sulfate-Free Shampoo

In a salon setting, stylists applied shampoo composition 1E to one half of consumers' heads of hair, massaged the shampoo into the hair thoroughly for 15 seconds, rinsed the hair for 15 seconds, the hair was combed, and then applied conditioner composition CND to the same half of the hair and left the conditioner composition on the hair for three minutes before thoroughly rinsing the hair. The same amounts of comparative shampoo composition C1-S and conditioner composition C1-C, commercially available and advertised as sulfate-free, were applied to the opposite side of the head of hair following the same procedure. The hair was then combed again, dried with a hair dryer, combed, and styled.

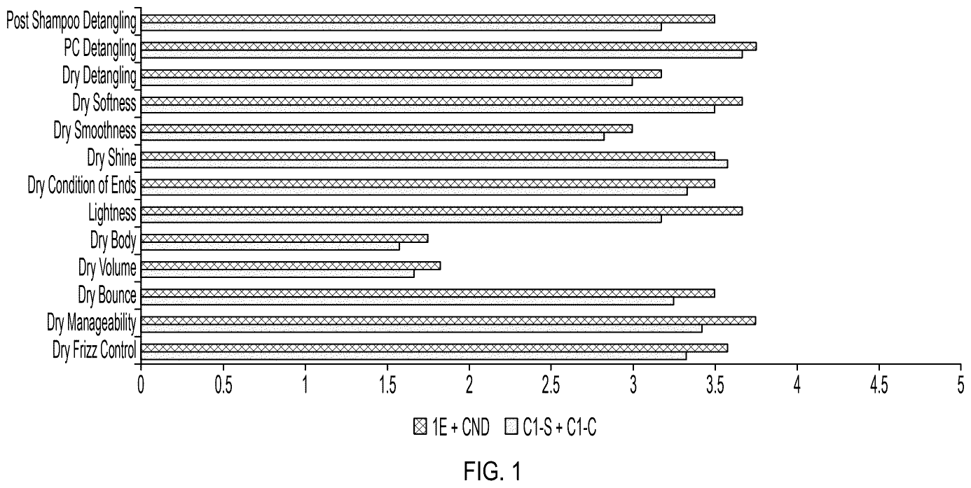

The stylists evaluated properties of the hair during and after the routine. All stylists found that the properties imparted hair treated with compositions 1E and CND were superior to those of hair treated with comparative shampoo composition C1-S and conditioner composition C1-C. The results are shown in FIG. 1. Example 1-1 therefore demonstrates that the combination of surfactants according to the disclosure is surprisingly more effective in the absence of sulfates at imparting cleansing and cosmetic benefits to hair than known sulfate-free shampoo compositions.

### Example 1-2—Foam Abundance Compared to Sulfate-Containing Shampoo

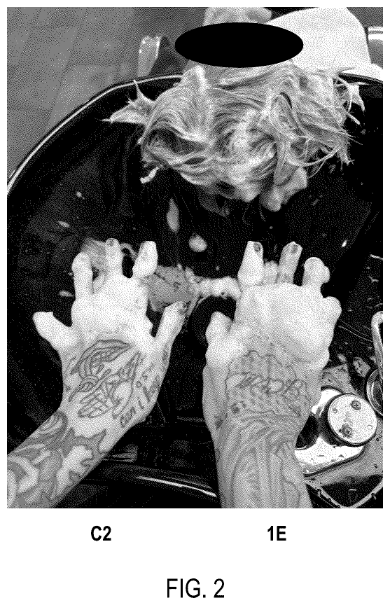

The abundance of foam generated with shampoo composition 1E, without sulfate surfactants, was compared to that of comparative shampoo composition C2 containing sulfate surfactants. After massaging equal amounts of composition 1E into one half of the head of hair and the comparative shampoo composition into the other half of the head of hair, the abundance and stability of foam generated was evaluated. In all cases, the abundance of foam generated with composition 1E according to the disclosure was superior to that of comparative shampoo composition C2. As can be seen in FIG. 2, the amount of foam on the stylist's right hand (inventive) is noticeably greater than the amount of foam on the left hand (comparative).

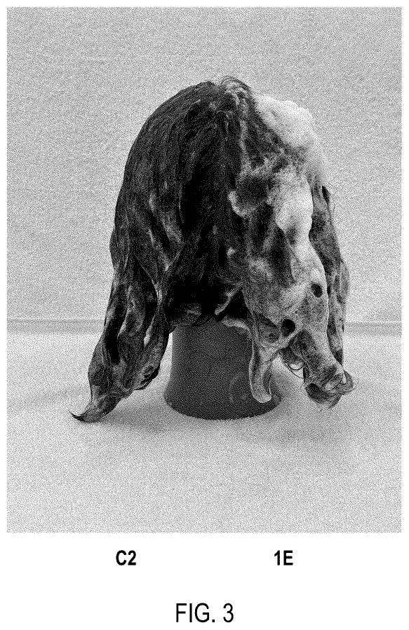

Foam abundance was separately evaluated in additional mannequin head studies, following the same procedure, with the same results. FIG. 3 shows a mannequin head with comparative shampoo composition C2 on the left side, and noticeably greater foam abundance generated by composition 1E on the right side.

The results of Example 1-2 are surprising since comparative shampoo composition C2 includes sulfate-based surfactants, which are known to provide good foaming. It therefore would have been expected that composition C2 would have outperformed composition 1E in terms of foam abundance.

### Example 1-3—Foam Stability Compared to Sulfate-Containing Shampoo

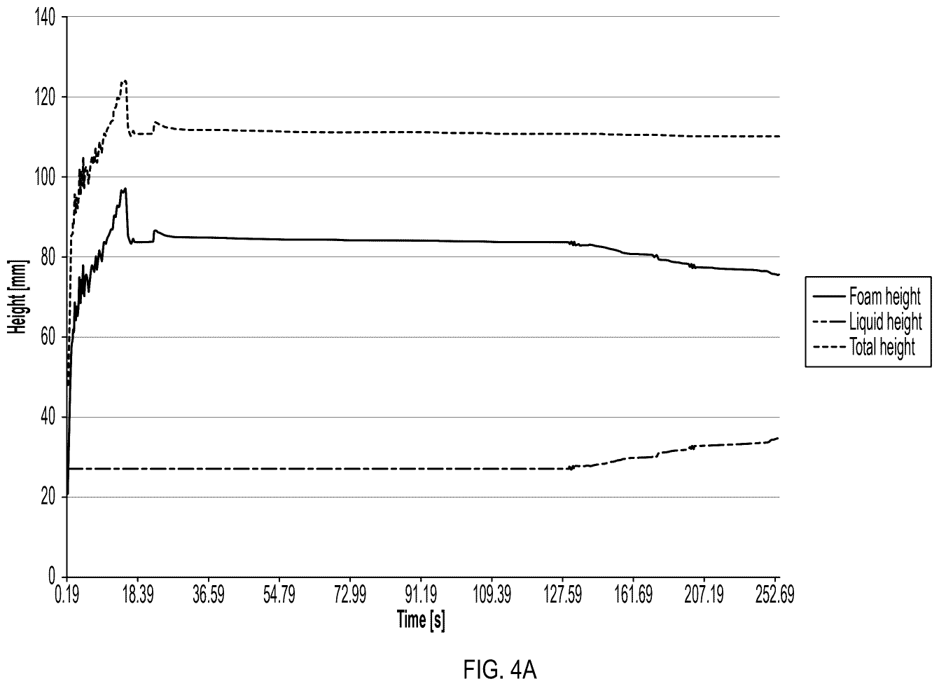

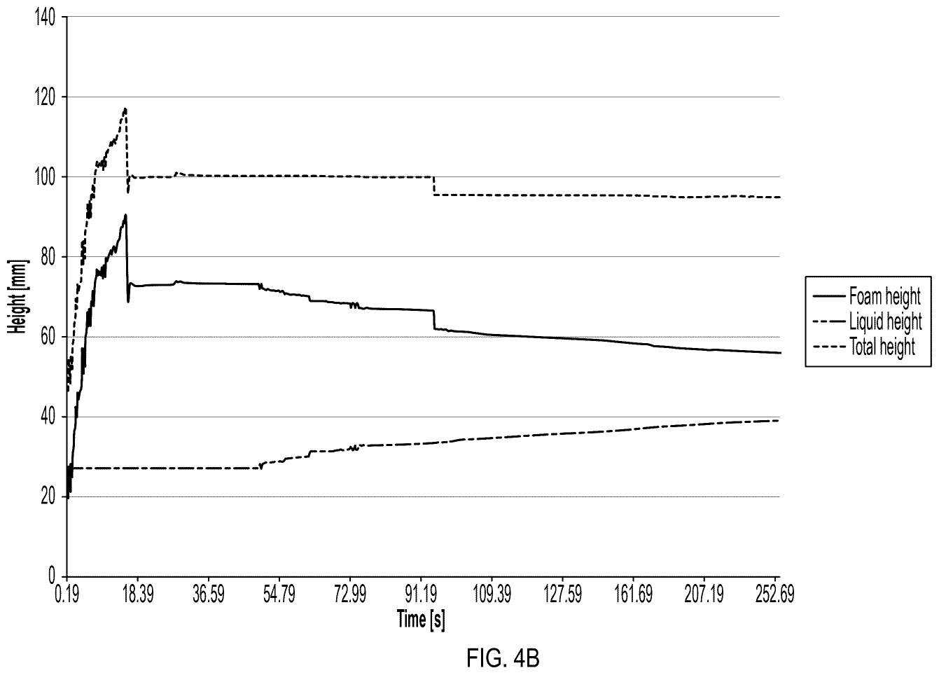

The stability of foam generated with shampoo composition 1E according to the disclosure was compared to the stability of foam generated by comparative shampoo composition C2 by measuring the height of foam generated over time using a Kruss DFA100 Dynamic Foam Analyzer. Samples were prepared using 5% of shampoo composition 1E or C2 and 95% deionized water, based on the total weight of the sample. The samples were dispersed in a cylinder and mixed using a vertical blade stirrer to generate foam for 15 seconds. The foam height, liquid height, and total height for each sample were measured over time. The results are shown in FIGS. 4A-4B.

As can be seen in FIG. 4A, the average total height of foam formed with composition 1E was 110.43 mm, while FIG. 4B shows that the average total height of foam formed with comparative shampoo composition C2 was 100.16 mm. The results of Example 1-3, which show an average difference in foam height over the same period of time of 10.27 mm, are surprising because a difference greater than 8 mm is considered significant. These results are even more surprising since comparative shampoo composition C2 includes sulfate-based surfactants, which are known to provide good foaming.

The results in Example 1 demonstrate that hair cleansed with compositions according to the disclosure has better cosmetic properties than hair cleansed with commercially-available sulfate-free shampoo, and that the foam generated by the inventive compositions is superior to that of commercially available sulfate-containing shampoo compositions. These results confirm that the combination of surfactants disclosed herein surprisingly provides cleansing, foaming, and cosmetic benefits expected from a shampoo composition containing sulfates, even in the total absence of sulfate-based surfactants.

### Example 2—Evaluation of Weight Ratios

To evaluate the impact that the weight ratio of the total amount of anionic surfactants to the total amount of amphoteric surfactants has on shampoo compositions according to the disclosure, and hair treated with the shampoo compositions, the additional shampoo compositions shown in Table 2A were prepared.

TABLE 2A

| | SHAMPOO | |
|---|---|---|
| | COMPOSITIONS | |
| | 2A | 2B |
| CERAMIDE AP, CERAMIDE NP, AND/OR | 0.0039 | 0.0039 |
| CERAMIDE EOP | | |
| XANTHAN GUM | 0.0049 | 0.0049 |
| CATIONIC SURFACTANT | 0.02 | 0.02 |
| ZINC PYRITHIONE | 1.02 | 1.02 |
| SODIUM LAURYL SULFOACETATE | 0.21 | 0.21 |
| ACRYLATES COPOLYMER | 0.42 | 0.42 |
| DISODIUM LAURETH SULFOSUCCINATE | 0.54 | 0.54 |
| PHYTOSPHINGOSINE AND CHOLESTEROL | 0.0027 | 0.0027 |
| COCAMIDE MIPA | 0.7 | 0.7 |
| COCO-BETAINE | 0.27 | 0.27 |
| SODIUM C14-16 OLEFIN SULFONATE | 5.7 | 2.28 |
| AMODIMETHICONE | 1.14 | 1.14 |
| POLYQUATERNIUM-10 | 0.8 | 0.8 |
| DISODIUM COCOAMPHODIACETATE | 0.63 | 0.63 |
| TRIETHYL CITRATE | 0.0017 | 0.0017 |
| TRIDECETH-6 | 0.1 | 0.1 |
| CARBOMER | 0.35 | 0.35 |
| SODIUM HYALURONATE | 0.01 | 0.01 |
| PPG-5-CETETH-20 | 1.0 | 1.0 |
| GLYCOL DISTEARATE | 1.35 | 1.35 |
| ADDITIVES* | <3 | <3 |
| SOLVENTS (water and non-aqueous solvents) | QS | QS |
| | to 100 | to 100 |

*preservatives, pH adjusters, stabilizers, consistency agents, electrolytes, antioxidants, emulsifiers

A study was performed using composition 1A from Table 1A and compositions 2A-2B from Table 2A. The weight ratio of each composition is shown in Table 2B.

TABLE 2B

| | 2A | 2B | 1A |
|---|---|---|---|
| Weight ratio of [total amount of first anionic | 9:1 | 4.5:1 | 3.3:1 |
| surfactant(s) + total amount of second anionic | | | |
| surfactant(s)]:amphoteric surfactant(s) | | | |

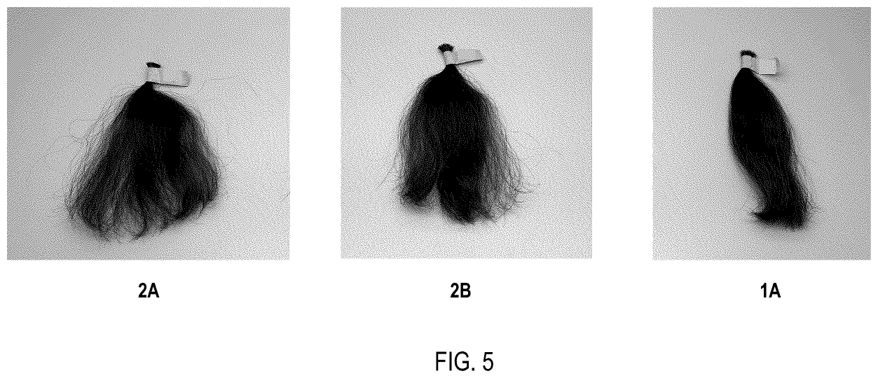

Equal amounts of shampoo compositions A, 2A, or 2B were massaged into separate swatches of Type 4 hair for 15 seconds, the hair was rinsed for 15 seconds, and then blown dry. During and after the process, various attributes of the shampoo, foam formed, and properties of the hair were evaluated on a scale of 1 (poor) to 5 (good). The results are shown in Table 20 and FIG. 5.

TABLE 2C

| | | | | |
|---|---|---|---|---|
| PROPERTY | 2A | 2B | 1A | OBSERVATIONS |

WET PROPERTIES

| | | | | |
|---|---|---|---|---|
| Foam flash | 1 | 2 | 3 | 1A foamed right away |
| Ease of lathering | 2 | 2 | 4 | 1A was easiest to distribute |
| Foam abundance | 1 | 2 | 3 | 2A did not produce abundant |
| | | | | foam; 2B had more foam than |
| | | | | 2A; 1A produced a stable, rich |
| | | | | foam |
| Foam stability | 1 | 2 | 3 | 2A foam was watery; 2B foam |
| | | | | eventually collapsed; 1A foam |
| | | | | was stable, creamy, and rich |
| Smoothness of foam | 1 | 3 | 4 | Hair felt creamy and smooth with |
| | | | | 1A |
| Ease of rinse | 4 | 4 | 4 | All easy to rinse |
| Wet detangling | 2 | 3 | 3 | 2B and 1A were easier to |
| | | | | detangle than 2A |

DRY PROPERTIES

| | | | | |
|---|---|---|---|---|
| Ease of blow drying | 4 | 3 | 4 | All easy to dry |
| Detangle | 1 | 2 | 4 | 1A easy to comb, no tangles; 2B |
| | | | | had more tangles; 2A had most |
| Softness | 1 | 3 | 4 | 2A felt rough; 2B softer than 2A; |
| | | | | 1A noticeably softer |
| Smoothness | 1 | 2 | 4 | 1A was noticeably smoothest |
| Condition of ends | 2 | 1 | 3 | 2A felt rough; 2B softer than 2A; |
| | | | | 1A noticeably softer |
| Frizz Control | 2 | 1 | 4 | 2A and 2B more frizzy than 1A |

As Table 20 and FIG. 5 show, the weight ratio of the total amount of anionic surfactants to the total amount of amphoteric surfactants impacts the properties of the shampoo composition, the foam generated, and the hair treated with the composition. This study demonstrates that as the weight ratio decreases, improvement in many desired properties and benefits is seen. Thus, the weight ratio of [total amount of first anionic surfactant(s)+total amount of second anionic surf actant(s)]:amphoteric surfactant(s) is preferably less than about 10:1, more preferably less than about 9:1, most preferably less than about 5:1, in order to achieve the desired advantageous benefits.

### Example 3—Additional Compositions

Compositions 3A-3D can also be prepared as shown in Table 3, and are likewise expected to provide similar foaming, cleansing, softness, frizz, and scalp care benefits.

TABLE 3

| | 3A | 3B | 3C | 3D |
|---|---|---|---|---|
| CERAMIDE AP, CERAMIDE NP, | 0.008 | 0.5 | 0.15 | 0.02 |
| AND/OR CERAMIDE EOP | | | | |
| BEHENTRIMONIUM | 0.05 | | | |
| CHLORIDE | | | | |
| HYDROXYPROPYL | 0.30 | | | 0.2 |
| CELLULOSE | | | | |
| MAIZE STARCH | | 1.00 | | |
| POLYQUATERNIUM-10 | | | 0.50 | |
| SODIUM COCOYL TAURATE | 1.00 | 1.00 | 0.75 | |
| DIOCTYL SODIUM | 2.00 | 2.50 | 1.50 | 1.00 |
| SULFOSUCCINATE | | | | |
| COCOBETAINE | 1.00 | 0.50 | | 0.75 |
| SODIUM C14-16 OLEFIN | 7.50 | 3.50 | | 1.00 |
| SULFONATE | | | | |
| AMMONIUM ALPHA-OLEFIN | | | 5.00 | 1.00 |
| SULFONATE | | | | |
| AMODIMETHICONE AND/OR | 1.0 | 1.5 | 0.50 | 0.10 |
| DIMETHICONE | | | | |
| CETEARYL ALCOHOL | | | 0.20 | 0.50 |
| DISODIUM | | | 1.50 | |
| COCOAMPHODIACETATE | | | | |
| DISODIUM | 2.50 | | 0.50 | 1.00 |
| CAPRYLOAMPHODI- | | | | |
| PROPIONATE | | | | |
| TRIDECETH-6 | 0.1 | 0.1 | 0.1 | 0.1 |
| CARBOMER | 0.35 | 0.35 | 0.35 | 0.35 |
| SODIUM HYALURONATE | 0.005 | | | 0.02 |
| PPG-5-CETETH-20 | 0.5 | 0.5 | 0.5 | 0.5 |
| GLYCOL DISTEARATE | 1.35 | 1.35 | 1.35 | 1.35 |
| ADDITIVES (preservatives, | <3 | <3 | <3 | <3 |
| vitamins, pH adjusters, stabilizers, | | | | |
| antioxidants, etc.) | | | | |
| SOLVENTS (water and non- | QS | QS | QS | QS |
| aqueous solvents) | TO 100 | TO 100 | TO 100 | TO 100 |

The above Examples demonstrate that shampoo compositions with scalp benefit agents having the combination of surfactants according to the disclosure are surprisingly effective at cleansing and providing desirable cosmetic properties to the hair, as well as generating abundant, thick, and creamy foam in sulfate-free shampoo compositions.

1. A composition for cleansing keratin materials comprising:
(a) a surfactant system comprising:
(i) at least one first anionic surfactant chosen from sulfosuccinates, sulfoacetates, acyl sarcosinates, salts thereof, or combinations of two or more thereof,
(ii) optionally at least one second anionic surfactant different from the first anionic surfactant,
(iii) optionally at least one amphoteric surfactant, and
(iv) optionally at least one nonionic surfactant;
(b) at least one scalp benefit agent chosen from ceramide AP, ceramide NP, ceramide EOP, and combinations of two or more thereof; and
(c) water,
wherein the composition is essentially free of sulfate-based surfactants.

2. The composition of claim 1, comprising at least one second anionic surfactant chosen from C4-C28 alkyl sulfonates, C8-C30 alkyl sulfosuccinates, isethionates, C8-C30 alkyl sulfoacetates, salts thereof, or combinations of two or more thereof.

3. The composition of claim 1, comprising at least one second anionic surfactant chosen from C10-C24 olefin sulfonates, or salts thereof.

4. The composition of claim 3, comprising at least one first anionic surfactant chosen from disodium laureth sulfosuccinate, sodium lauryl sulfoacetate, sodium lauroyl sarcosinate, or combinations of two or more thereof.

5. The composition of claim 1, comprising at least one first anionic surfactant chosen from C6-C30 alkyl sulfosuccinates, C6-C30 alkyl sulfoacetates, acyl sarcosinates, salts thereof, or combinations of two or more thereof.

6. The composition of claim 1, wherein the total amount of anionic surfactants present in the composition ranges from about 0.5% to about 15% by weight, relative to the total weight of the composition.

7. The composition of claim 1, comprising at least one amphoteric surfactant chosen from betaines, C8-C18 alkyl sultaines, C8-C18 alkyl amphoacetates, amphopropionates, salts thereof, or combinations of two or more thereof.

8. The composition of claim 1, wherein the composition is essentially free of cocamidopropyl betaine.

9. The composition of claim 1 comprising at least one amphoteric surfactant, wherein the total amount of amphoteric surfactants present in the composition ranges from about 0.5% to about 10% by weight, relative to the total weight of the composition.

10. The composition of claim 9, wherein the weight ratio of the total amount of first and second anionic surfactant(s) to the total amount of amphoteric surfactant(s) ranges from about 1:1 to about 8:1.

11. The composition of claim 1, comprising at least one nonionic surfactant, wherein the total amount of nonionic surfactants present in the composition ranges from about 0.5% to about 15% by weight, relative to the total weight of the composition.

12. The composition of claim 1, wherein the total amount of scalp benefit agents chosen from ceramide AP, ceramide NP, and ceramide EOP ranges from about 0.0001% to about 1% by weight, relative to the total weight of the composition.

13. The composition of claim 1, comprising at least one conditioning agent chosen from cationic conditioning agents, silicone compounds, non-silicone fatty compounds, or combinations thereof.

14. The composition of claim 1, further comprising at least one anti-dandruff active agent.

15. A composition for cleansing keratin materials comprising:
(a) a surfactant system comprising:
(i) at least one first anionic surfactant chosen from C8-C30 alkyl sulfosuccinates, C8-C30 alkyl sulfoacetates, acyl sarcosinates, salts thereof, or combinations of two or more thereof,
(ii) optionally at least one second anionic surfactant chosen from C4-C28 alkyl sulfonates, salts thereof, or combinations of two or more thereof,
(iii) at least one amphoteric surfactant, and
(iv) optionally at least one nonionic surfactant;
(b) at least one scalp benefit agent chosen from ceramide AP, ceramide NP, ceramide EOP, and combinations of two or more thereof;
(c) water;
(d) optionally at least one anti-dandruff active agent; and
(e) optionally at least one conditioning agent,
wherein the weight ratio of the total amount of first and second anionic surfactants to the total amount of amphoteric surfactant(s) ranges from about 1:1 to about 6:1; and
wherein the composition is essentially free of sulfate-based surfactants and cocamidopropyl betaine.

16. The composition of claim 15, comprising at least one anti-dandruff agent chosen from salts of pyrithione.

17. The composition of claim 15, wherein the total amount of scalp benefit agents chosen from ceramide AP, ceramide NP, and ceramide EOP ranges from about 0.0001% to about 1% by weight, relative to the total weight of the composition.

18. A method for cleansing keratin materials comprising:
applying a composition to the keratin materials, the composition comprising:
(a) a surfactant system comprising:
(i) at least one first anionic surfactant chosen from sulfosuccinates, sulfoacetates, acyl sarcosinates, salts thereof, or combinations of two or more thereof,
(ii) optionally at least one second anionic surfactant different from the first anionic surfactant,
(iii) optionally at least one amphoteric surfactant, and
(iv) optionally at least one nonionic surfactant;
(b) at least one scalp benefit agent chosen from ceramide AP, ceramide NP, ceramide EOP, and combinations of two or more thereof; and
(c) water,
wherein the composition is essentially free of sulfate-based surfactants, and
rinsing the composition from the keratin materials.

19. The method of claim 18, wherein the composition comprises at least one amphoteric surfactant, and the weight ratio of the total amount of first and second anionic surfactant(s) to the total amount of amphoteric surfactant(s) ranges from about 1:1 to about 8:1.

20. The method of claim 18, wherein the composition is a shampoo composition and the keratin materials are hair.
